(19) United States
(12) Patent Application Publication
(10) Pub. No.: US 2013/0196358 A1
(43) Pub. Date: Aug. 1, 2013

(54) METHOD FOR THE DIAGNOSIS OF AMYOTROPHIC LATERAL SKLEROSIS

(71) Applicant: Bert Steffan, Koln (DE)

(72) Inventor: Bert Steffan, Koln (DE)

(21) Appl. No.: 13/697,917

(22) PCT Filed: May 11, 2011
(86) PCT No.: PCT/EP2011/057638

§ 371 Date: Mar. 11, 2013

Related U.S. Application Data
(60) Provisional application No. 61/334,716, filed on May 14, 2010.

(30) Foreign Application Priority Data
May 14, 2010 (EP) 10005076.4

(51) Int. Cl. G01N 33/48; G01N 33/92; G01N 33/68; C12Q 1/06

(52) U.S. Cl. CPC G01N 33/48; C12Q 1/06; G01N 33/92; G01N 33/6896; G01N 33/6854
USPC 435/17; 435/39; 436/71; 436/86; 73/61.41; 324/309

(57) ABSTRACT

Subject of the invention is a method for the diagnosis of amyotrophic lateral sclerosis, comprising the steps of
a) providing a sample from a patient,
b) determining in the sample the level and/or activity of at least one marker, which is indicative of a glucose consumption disorder,
c) comparing the level and/or activity to a known standard and
d) deducing whether the patient has, or is susceptible to, amyotrophic lateral sclerosis.

Subject of the invention are methods for the diagnosis of amyotrophic lateral sklerosis (ALS).

### STATE OF THE ART

Amyotrophic lateral sclerosis (ALS, also referred to as Lou Gthrig's disease) is a form of motor neuron disease (MND). ALS is a progressive, fatal, neurodegenerative disease caused by the degeneration of motor neurons, the nerve cells in the central nervous system that control voluntary muscle movement.

ALS affects approximately one in every 20,000 individuals, with an average age onset of 50 to 55 years. ALS is characterized by rapidly progressive degeneration of motor neurons in the brain, brainstem and spinal core. The median survival of patients from time of diagnosis is approximately five years. The cause of the disease is not known, and an effective treatment was not yet found. The search for treatments and causes is difficult, because at least three individual types of the disease are observed. These are sporadic ALS (sALS), familiar ALS (fALS) and familiar SOD1-ALS. Further, within each of the types there are individual characteristics.

Definite and reliable biomolecular parameters for the diagnosis of ALS do not exist.

ALS was not yet correlated clearly to genetic markers. Mutations in the SOD1 gene with ALS allow an attribution to this subcategory, but recent findings suggest that they have a rather incidental character, as these mutations are not necessarily connected to the appearance of the disease in humans. Lately, other genetic parameters were discussed, which are TDP43, mutations in the spastin gene, transthyretin and cystatine C. None of these parameters could so far be connected strictly to ALS from a diagnostic point of view. Thus presently no genetic correlation for ALS exists, in contrast to other neurodegenerative diseases such as Huntington disease.

Many attempts have thus been made to determine biomarkers in body fluids of patients. Turner et al., 2009, provide a review about the literature relating to the diagnosis of ALS on the basis of biomarkers, especially based on CSF, blood and urine. The authors summarize the state of the art as follows: “Although there are few diseases that mimic ALS and even fewer treatable or remitting mimics, there is no diagnostic test for ALS, and confident diagnosis is mainly based on clinical assessments and relies on the detection of upper motor neuron (UMN) and lower motor neuron (LMN) signs in the limb and/or bulbar territories, together with a history of progression of symptoms” (page 94, right column). Presently, diagnostic security can be achieved earliest within one year after the first signs through clinical methods, such as electromyography.

As summarized by Turner at al., various publications tried to correlate biomarkers from blood with ALS. Findings included raised concentrations of tyrosine in the serum and glutamate in the plasma, as well as decreased concentrations of large neutral amino acids in the plasma, particularly in patients who had more affected bulbar territories compared with controls. With regard to potential cytokine biomarkers, raised concentrations of interleukin 6 have been shown in the serum of patients with ALS, with 73% sensitivity and 91% specificity.

Patten et al, 1978, studied the relation of amino acid levels to ALS. Levels of branched chain amino acids and valine increased with longer duration of disease, and levels of aspartate and acidic amino acids were found to increase. A study of amino acid levels in ALS patient plasma was published by Ilzecka, 2003. However, the studies relating to amino acid levels are on a theoretical level and a reliable diagnosis of ALS based on amino acid levels was neither disclosed nor developed subsequently.

In other studies, lipid metabolism has been suggested to affect progression and survival in ALS. A high ratio of low density to high-density lipoprotein was associated with raised survival times.

Mitchell et al., 2009, studied 27 biomarkers from cerebrospinal fluid specimen (CSF, Liquor cerebrospinalis), which might be suitable to differentiate ALS from other neurobiological diseases. The authors concluded that 5 of the 27 tested substances would be suitable to distinguish ALS from other neuronal diseases. These are interleukin 10 (IL-10), IL-6, IL-2, IL-15 and GM-CSF (Granulocyte-Monocyte Colony Stimulating Factor). However, these are essentially inflammation markers, which can also be indicative of numerous other diseases, which are not neurobiological.

Further biomarkers, which potentially might be used in the diagnosis of ALS, are disclosed in WO 2007/100782. However, a large number of potential markers are provided without information about their significance and identity.

In a comparative study, which included a post-mortem analysis of the CSF, biomarkers from recently diagnosed patients (but without controls) seemed to be unchanged in separate post-mortem cases of different patients (Ranganathan, 2009). This finding highlights the need to identify biomarkers that change with disease progression.

The absence of definite and reliable markers for the diagnosis of ALS is highly problematic, because it is difficult to distinguish ALS from other neurodegenerative diseases with similar symptoms. Clinically, the overall symptoms of ALS are highly similar to other diseases, such as rare symptomatic forms of spinal muscle atrophies associated with malignoma, dysproteinamia, intoxications or sugar metabolism disorders. Spinal muscle atrophies are also partial symptoms in diseases such as Parkinson-dementia-complex, Creutzfeld Jacob disease, Friedreich-ataxie, intraspinal processes, orthostatic hypotony, and others. A differential diagnosis against motoric polyneuropathie or poliomyelitis for ALS is only possible after liquor, EMG and CT examination and muscle biopsy. In the presence of bulbary symptoms, components of a pseudo bulbary paralysis can be involved.

In summary, the diagnosis of ALS and the distinction from other diseases is highly complicated. Extensive tests are necessary, which are incriminating for the patient, for example in the case of muscle biopsy.

### PROBLEM UNDERLYING THE INVENTION

The problem underlying the invention is to provide a method for the diagnosis of ALS. The method shall overcome the above-mentioned problems. The method shall be reliable and specific.

A specific problem underlying the invention is to provide a method for the diagnosis of ALS, which can be carried out relatively simple. The method shall be applicable with samples, which are readily available. The method shall only require a low number of process steps.

Further, the invention shall provide a method for the diagnosis of ALS, which is convenient for the patient. It shall not be necessary to carry out examinations, which are painful and incriminating for the patient, such as muscle biopsy.

It is a further problem of the invention to provide a method for the diagnosis of ALS, which can distinguish ALS from other neurodegenerative diseases. The method shall also distinguish ALS from other diseases, which have similar clinical symptoms as ALS. The method shall be useful in monitoring the progression of ALS or monitoring a therapy against ALS.

### DISCLOSURE OF THE INVENTION

Surprisingly, the problem underlying the invention is solved by methods according to the claims. Subject of the invention is a method for the diagnosis of amyotrophic lateral sclerosis, comprising the steps of
a) providing a sample from a patient,
b) determining in the sample the level and/or activity of at least one marker, which is indicative of a glucose consumption disorder,
c) comparing the level and/or activity to a known standard and
d) deducing whether the patient has, or is susceptible to, amyotrophic lateral sclerosis.

According to the invention, the diagnosis of amyotrophic lateral sclerosis is correlated to the determination of symptoms of a glucose consumption disorder. In a glucose consumption disorder, the metabolic consumption of glucose is disturbed. In humans, the metabolic consumption of glucose comprises conversion into pyruvate during glycolysis and subsequent degradation of pyruvate in the citric acid cycle. When glucose consumption is impaired or disturbed in the human body, often glucose levels in body liquids, especially blood, are increased or decreased or show irregular variations.

Glucose levels and levels of markers relating to glucose consumption are routinely measured when examining the blood or urine of patients in the diagnosis of various diseases. However, the present invention for the first time links the diagnosis of ALS to symptoms of a glucose consumption disorder. The present invention is based on the unexpected finding that ALS coincides with symptoms of a glucose consumption disorder. To the knowledge of the inventors, it was not known in the art to deduce systematically and causative from symptoms of a glucose consumption disorders whether a patient has, or has not, ALS. The present inventive method is thus distinct from any potential prior art disclosure, in which an altered level of glucose or other metabolites was merely observed without realizing the diagnostic impact for ALS, or in which an altered level of glucose or other metabolites might have been attributed to a secondary medical condition of an ALS patient.

In a preferred embodiment of the invention, the glucose consumption disorder is a ketonuria, preferably a ketoacidosis.

A ketonuria is a medical condition, in which increased levels of ketones are found in body liquids. Specifically, a ketonuria is associated with the increased or at least altered presence of ketone bodies in body liquids, such as urine, blood or CSF. Ketone bodies are water soluble metabolites, which are by-products when fatty acids are metabolically broken down for energy generation. The three main ketone bodies are acetone, acetoacetate and beta-hydroxy butyric acid. Betahydroxy butyric acid is not a ketone, but a carboxylic acid. A ketonuria is frequently associated with type I diabetes mellitus. Production of ketone bodies during ketonuria is a typical response to a shortage of glucose, wherein fatty acids are consumed as an alternative source of energy. In healthy individuals, ketones are formed in the liver and completely metabolised, so that only negligible amounts appear in body liquids, such as urine, blood or CSF.

A ketoacidosis is a type of metabolic acidosis, which is associated with high concentrations of ketone bodies. In a ketoacidosis, the production of ketone bodies from fatty acids is accompanied by a pH decrease. In general, a ketoacidosis is most common in type I diabetes mellitus.

A lactate alkalosis is usually detectable in blood and/or urine. This physiological condition is characterized by a high pH in blood (alkalosis), which is accompanied by high levels of lactate. For example, serum pH can be above 7.4 or 7.5 and lactate levels can be above 4 mmol/l.

In a preferred embodiment of the invention, the sample is a body liquid, such as cerebrospinal fluid (CSF), urine or blood, or a fraction of any of these samples.

The use of body liquids in the diagnosis of ALS is highly preferred, because they are readily available. Thus the examination is simple and convenient for the patient. Specifically, in the case of ALS the testing of body liquids is more convenient than invasive diagnostic methods, such as muscle biopsy.

In the prior art, various biomarkers from blood, urine and CSF have been studied for a potential significance in ALS diagnosis. However, a direct correlation to a glucose consumption disorder, specifically a ketonuria or ketoacidosis has not yet been made. According to the invention, it was found that when determining systematically for a patient suspected of ALS, whether typical symptoms of a glucose consumption disorder, such as ketonuria, ketoacidosis or lactate alkalosis are found, a specific and highly reliable diagnosis of ALS can be made. Specifically, it was found that a distinction of ALS from other neurodegenerative diseases is possible. Further, ALS can be distinguished from other diseases, which have similar clinical symptoms, on a molecular level.

In a preferred embodiment, the sample is cerebrospinal fluid (CSF). In this embodiment, it is preferred that the disorder is associated with a dysfunction in the brain, specifically a dysfunction of motor neurons.

Biomarkers in blood, that have been studied in the prior art, do not offer improved sensitivity and specificity compared with clinical diagnosis in patients with ALS. The diagnostic method of the invention is efficient, because it links the diagnosis of the ALS to changes in specific physiological pathways. In other words, the invention is based on the finding that a specific physiological pathway is affected, when a patient has ALS. In view of this insight, it is at the skilled person's discretion which markers and how many markers are examined for a specific patient. The present inventive method provides a more reliable diagnosis of ALS compared to methods known in the art, in which isolated markers, the significance of which is not understood, are determined.

The present inventive method allows a diagnosis whether a patient has, or is susceptible to, ALS. The inventive method can be used for monitoring the progression of ALS in a patient. The inventive method can also be used for monitoring a therapy of an ALS patient. The inventive method can also be used for distinguishing ALS from other neurodegenerative diseases or from other diseases having similar clinical symptoms as ALS.

The sample can be a fraction of a body liquid. For example, the sample can be blood serum or plasma or a fraction thereof. The CSF can be obtained by known methods, for instance lumbar puncture.

The sample can be a body fluid or a fraction thereof. For example, body fluids, such as blood or CSF can be subjected to a prior separation of proteins.

In a preferred embodiment of the invention, the marker is a metabolic marker (metabolite). Metabolites are intermediates and end-products of metabolism. Usually, metabolites are small molecules. For example, metabolites may have molecular weights below 2,000 or below 1,000 Dalton. Preferably, metabolites are not functional proteins.

According to the invention, in step b) the level and/or activity of at least one marker is determined. Especially in the case of metabolic markers, it is frequently more convenient to determine the level of the marker than the activity. Determining the “level” also relates to determining a ratio of two markers.

According to the invention, it is preferred that the level of more than one marker is determined. Preferably, the level and/or activity of at least two, at least three, at least four, at least five or at least six markers is determined. In preferred embodiments, the levels and/or activities of 2 to 20 markers, or of 3 to 15 markers, or of 4 to 10 markers are determined. In general, the skilled person understands that the combination of a few markers of high significance will allow a strong correlation with ALS.

In a preferred embodiment of the invention, the sample is cerebrospinal fluid (CSF) and the glucose consumption disorder is a ketoacidosis, and/or

the sample is urine and the glucose consumption disorder is a phenylketonuria, and/or
the sample is blood and the glucose consumption disorder is a lactate alkalosis.

In a preferred embodiment of the invention, at least one, preferably at least 2, at least 3 or at least 4 markers are selected from the group consisting of acetone, acetoacetone, phenyl compounds which are not peptides, alkenales, pyruvate, catecholamines, preferably adrenalin and noradrenalin, glucose, lactate, citrate, creatine, phosphocreatine, insulin, cortisol and glutathion. Preferably, the ketone bodies are acetone or acetoacetone. It was found that these markers are significantly increased, especially in CSF. Preferably, the phenyl compounds are low molecular weight compounds, preferably phenylacetate, phenylpyruvate or phenyllactate. In a specific embodiment, the phenyl compounds do not comprise phenylalanine

In a preferred embodiment of the invention, the sample is cerebrospinal fluid (CSF) the marker is at least one, preferably 2 or 3, selected from the group consisting of ketone bodies, especially acetone, acetylacetone, and cyclopropane. In these embodiments relating to CSF, the glucose consumption disorder is preferably a ketoacidosis. According to the invention, it was found that especially levels of acetone and acetylacetone in CSF are significantly increased. It is highly preferred that the level of at least acetone or acetoacetone, or both of acetone and acetoacetone, is determined. The third ketone body, which is 13-hydroxybutyric acid, was also found to be altered significantly. However, the level was decreased in ALS patients CSF.

It is known in the art that common glucose consumption disorders, such as diabetes type I, are often detectable by metabolic markers in blood or urine. Specifically, in the case of diabetes mellitus type I, symptoms of a ketonuria or ketoacidosis are observed in blood or urine. However, patients having diabetes mellitus type I do not have symptoms of ketonuria or ketoacidosis in CSF. Thus, when the present inventive method is carried out with CSF, it can be excluded that a false positive result is obtained, because the patient has a different glucose consumption disorder, such as diabetes type I. Thus the use of a CSF sample in the inventive diagnostic method renders the diagnosis highly specific. The present inventive method is thus also useful for distinguishing ALS from diabetes mellitus, specifically type I.

Although it is advantageous according to the invention to determine symptoms of a glucose consumption disorder, specifically a ketonuria or ketoacidosis, in CSF, also blood or a fraction thereof or urine can be examined in the inventive method for these disorders. Markers such as acetone or acetoacetone were found to be less pronounced in blood or urine, but still detectable at relevant levels.

In another preferred embodiment of the invention, the sample is urine and at least one marker, or at least 2 or at least 3 markers are preferably selected from the group consisting of a phenyl compounds, preferably phenylacetate, phenylpyruvate or phenyllactate, alkenales and pyruvate. In these embodiments, the glucose consumption disorder is preferably a phenylketonuria. The alkenales can be typical metabolites, such as 2-nonenale, or alkenale derivatives, such as hydroxy nonenale. Specifically, it is preferred that the level of at least one, at least 2 or at least 3 phenyl compounds is determined. Although the examination of urine is preferred for these embodiments, the method is also applicable with blood or a fraction thereof.

In a preferred embodiment of the invention, the sample is blood or a fraction thereof and at least one marker, preferably at least 2 or at least 3 markers are selected from the group consisting of catecholamines, preferably adrenalin and noradrenalin, phenyl compounds, preferably phenylalanine, glucose, lactate, citrate, creatine, phosphocreatine, insulin, cortisol and glutathion. In these embodiments, the glucose consumption disorder is preferably a lactate alkalosis. Although the examination of blood or a fraction thereof is preferred for these embodiments, the method is also applicable with urine.

In a preferred embodiment of the invention, in step (d) it is deduced that the patient has, or is susceptible to, amyotrophic lateral sclerosis, when the level and/or activity of the markers deviates at least 10% from the known standard. Preferably, the level of the marker deviates at least 10%, more preferably at least 20%, at least 30% or at least 50% from the known standard. According to the invention, a “known standard” can be a known average value from a pool of healthy persons. Such standards are known in the art. Further, the known standard can be a reference from a single person. In another embodiment, the known standard is a value of the same patient, which was determined previously. For example, when comparing values of the same person, the progression of ALS can be monitored over time. In another embodiment of the invention, the known standard is a reference value of a person known to have ALS, or of a pool of persons known to have ALS.

In a preferred embodiment of the invention, the method comprises additionally determining in the sample the level and/or activity of at least one protein and/or cell, and/or the pH.

Specifically, it is determined according to the invention whether the pH of CSF is lowered, for example to a value below 7.5, preferably below 7.3 or below 7.2. Specifically, it is determined according to the invention whether the pH of blood is increased, for example to a value above 7.4, preferably above 7.5.

In a preferred embodiment of the invention, the enzymes and/or cells are selected from the group consisting of ANA, CK-Nac, CD3/T-cells, LDL, HDL, leucocytes, neutrophiles, monocytes, erythrocytes and amyloid A. Preferably, at least 2 or at least 3 of these markers are examined. Further markers, which have some correlation to ALS, can be used according to the invention to complement the inventive diagnostic assay.

Another subject of the invention is a method for the diagnosis of amyotrophic lateral sclerosis, comprising the steps of
a) providing a sample from a patient,
b) determining in the sample the level and/or activity of at least one metabolic marker,
c) comparing the level and/or activity to a known standard and
d) deducing whether the patient has, or is susceptible to, amyotrophic lateral sclerosis,
   wherein the at least one marker, preferably at least 2 or at least 3 markers are selected from the group consisting of acetone, acetoacetone, phenyl compounds which are not peptides, alkenales, pyruvate, catecholamines, preferably adrenalin and noradrenalin, glucose, lactate, citrate, creatine, phosphocreatine, insulin, cordsol and glutathion. Preferably, the ketone bodies are acetone or acetoacetone. Preferably, the phenyl compounds are low molecular weight compounds, preferably phenylacetate, phenylpyruvate or phenyllactate. In this method of the invention, the body liquids, markers, samples, marker levels etc. are selected as outlined above.

Preferably, the sample is cerebrospinal fluid (CSF) and the marker is selected from ketone bodies, especially acetone or acetylacetone, and cyclopropane. Preferably, the sample is urine or blood and wherein the marker is selected from the group consisting of a phenyl compounds, preferably phenylacetate, phenylpyruvate or phenyllactate, alkenales and pyruvate. Preferably, the sample is blood or urine and the marker is selected from the group consisting of catecholamines, preferably adrenalin and noradrenalin, phenyl compounds, which are not peptides, preferably phenylalanine, glucose, lactate, citrate, creatine, phosphocreatine, insulin, cortisol and glutathion.

In the following, markers which are preferred according to the invention and relevant levels of the markers are described in detail. Additional information is provided in the working examples further below. In CSF, urine and blood, the observation of the following markers is preferred (“+” indicates an increase and “−” indicates a decrease compared to a known standard.

| CSF (Liquor): | |
|---|---|
| Acetone: | +(10-60)%, preferably at least more than 10%, more |
| | than 20% or more than 50% |
| Acetoacetone: | +(10-60)%, preferably at least more than 10%, more |
| | than 20% or more than 50% |
| Cyclopropane: | +(10-60)%, preferably at least more than 10%, more |
| | than 20% or more than 50% |
| β-Hydroxybutyric | — |
| acid | |
| Urine: | |
| Phenylacetate: | +(20-150)%, preferably at least more than 10%, more |
| | than 30% or more than 100% |
| Phenylpyruvate: | +(20-150)%, preferably more than 10%, more than |
| | 20% or more than 50% |
| Phenyllactate: | +(20-150)%, preferably more than 20%, more than |
| | 50% or more than 100% |
| Pyruvate: | +(10-50)%, preferably at least more than 10%, more |
| | than 20% or more than 50% |
| Alkenals: | +(10-60)%, preferably at least more than 10%, more |
| | than 20% or more than 50% |
| Citrate: | +20%, preferably +30% |
| Blood: | |
| Catecholamines: | |
| Adrenalin: | +(20-70)%, preferably at least more than 10%, more |
| | than 20% or more than 50% |
| Noradrenalin: | +(10-80)%, preferably at least more than 10%, more |
| | than 20% or more than 50% |
| Phenylalanine: | +(20-80)%, preferably at least more than 10%, more |
| | than 20% or more than 50% |
| Glucose: | fluctuant 40-120, deviation from a reference standard |
| | of more than 20% for at least 50% of time |
| Insulin: | Fluctuant, from not detectable to 2x, deviation from a |
| | reference standard of more than 20% for at least 50% |
| | of time |
| Cortisol: | >17x |
| Lactate: | >3x, preferably >4x; preferably >4 or >5 mmol/1 |
| Citrate: | +20%, preferably +30% |
| Glutathione: | −(10-40)%, preferably more than 10% decrease or |
| | more than 20% decrease |
| IGF-1: | <150 mg/ml |
| ANA: | >1:150 titre |
| CK-Nac: | >190 U/l |
| CD3/T-Cells: | <400 FI mean |
| Amyloid A: | >10 |

Ratio citrate/creatine, creatinine, phosphocreatine: 1:1
pH-value blood: >7.52
pH-value CSF: <7.24

Without being bound to theory, it is assumed that overall pattern of marker levels in CSF, urine and blood can be explained as follows. A primary cause of ALS seems to be a glucose consumption disorder located, at least primarily, in the brain. Probably, the origin of the disorder is a malfunction in the motor cortex. As a consequence, an alternative energy production pathway is activated, which is increased fatty acid oxidation in the brain. As a result, acetone and acetoacetone accumulate in the CSF. These and other metabolites transmit a signal, that the glucose production is impaired. Thus the body tries to increase glucose production by known means, for example increased production of catecholamines and abnormal insulin. This reaction is comparable to a stress reaction, when the body produces adrenalin at increased levels in order to provide a temporarily high glucose level. However, in ALS the dysfunction in the brain cannot be reversed by these responses and thus the aberrant pattern is maintained. The high fluctuation of the glucose and insulin level seems to indicate that an auxiliary pathway is activated and deactivated without resulting in a normal glucose balance. Further, abnormal insulin levels seem to be a result of the disturbed glucose metabolism. An increased level of lactate seems to indicate that certain regions in the body, in this case presumably the motor neurons and the anterior horn cells, have switched to energy production from lactate instead of normal glucose based energy production.

The inventive pattern has some similarity to known processes, which are associated with temporarily increased fatty acid breakdown, especially during hunger or stress. However, the present method easily distinguishes ALS from these states when taking care that the patient was not subjected to stress or hunger when the sample was taken. Further, the aberrant values describes herein are not observed temporarily, but over extended times. Thus in a preferred embodiment of the invention, at least 2, preferably at least 3 or at least 4 probes from the same patient are examined, which were taken at different time points, for example within 6 or 24 hours or within 1 week or 1 month.

In a preferred embodiment of the invention, in step (b) the level of the marker, preferably the metabolic marker is determined by NMR spectroscopy, specifically 1H-NMR spectroscopy. Preferably, the 1H-NMR spectroscopy is one-dimensional. However, it is also possible to use 2-D NMR. Preferably, high-resolution NMR is used. For example, a one dimensional ¹H-spectrum can be obtained at 500 or 360 MHz at 25° C. In general, the analysis by NMR is preferred, because the method requires little or no pre-treatment of samples. Further, a large number of metabolites can be visualised in one single probe. High-resolution NMR has been used in monitoring progression and outcome of other diseases, such as schizophrenia (Holmes et al., 2006). In NMR diagnosis, the samples may be pre-treated by known methods, for example incubation with D₂O. The amount of marker in the sample is deducible from the peak area. The examination of body liquids, such as CSF, by NMR spectroscopy, has been described in the art. For example, Petroff et al., 1986, provide a summary of methods and devices for studying CSF components by high-resolution NMR. Another study of CSF by high-resolution NMR is disclosed by Wevers et al., 1995. In these publications, reference values for a large number of metabolites are disclosed. In a specific embodiment of the invention, samples are prepared and analysed as disclosed in one of these documents.

The present invention is based on studies of liquor, blood and urine samples of ALS patients with high-resolution NMR spectroscopy. Without being bound to theory, the overall findings are indicative of a glucose consumption disorder in the brain, specifically motor cortex. The glucose consumption disorder in the motor cortex is associated with a performance deficit in motor neurons 1 and 2. This is confirmed by PET-CT (Positron-Emission-Tomography), which indicates diminished glucose turnover in these areas of the brain. As a consequence, in these neuronal areas an alternative pathway is activated, through which energy supply to cells is provided by fatty acid cleavage. Subsequently, acetoacetate-S-coenzyme A enters the brain and contributes to energy supply of neuronal cells by conversion to acetate-S-coenzyme A. This alternative energy supply pathway is observed in healthy individuals during fasting or when glucose levels are strongly reduced for extended times.

In the inventive method, it is preferred that it is determined whether a lactate alkalosis is present in blood and/or urine. According to the invention, it was found that the level of lactate is unusually high in blood and urine. Further, the pH of blood was found to be unusually high. Thus, the patients have symptoms of a lactate alkalosis. In other diseases, an increased level of lactate in blood is often accompanied by a decreased pH. Such physiological states are referred to as lactate acidosis.

Some phenomena as described above resemble those observed for diabetes (diabetic polyneuropathy). During progression of diabetes, the formation of ketone bodies and the disturbance of the insulin signal pathway result in symptoms, which are similar to those described herein. However, during the present analysis of blood or urine no hint was found of diabetes type I. Thus, without being bound to theory, it is assumed that the glucose regulation in the body seems to be mostly intact. The preliminary malfunction seems to be located in the motoric nervous system, and herein specifically in pyramidal cells of the motor cortex. At least, this seems likely in view of the large numbers of ketone bodies found in the CSF, which result in a low pH of the liquor (around 7.23). As a result, high lactate values were observed in blood and urine. In blood, an increased pH (about 7.52 to 7.6) seems to indicate a metabolic alkalosis, which could induce symptoms of ALS, such as unnatural hand position.

In summary, the following processes seem to coincide, which cause a performance decrease of motoric neurons: excess acetoacetate-SCoA is not only converted to acetal-SCoA and used for subsequent energy production, but is also converted to ketone bodies, which induce a pH decrease and subsequent decrease of metabolism efficiency. Further, ketone bodies interact with cellular proteins and impair their regular function. The metabolic decrease results in an increased activity of lipoxygenases, which convert fatty acids for energy consumption. Alkenales, further ketone bodies and ROS are produced, which negatively affect the metabolism further. When lipoxygenases affect mitochondrial fatty acids, additional subsequent reactions, which could induce apoptotic processes, may occur.

### FIGURES

FIG. 1: 1H-NMR-spectra comparison: liquor of ALS patient (upper curve) and liquor of healthy individual (lower curve), pyruvate standard (second line from bottom, with single peak at about 2.375), acetone standard (second line from above, single signal at about 2.230), acetoacetate standard (upper line, single signal at about 2.285).

FIG. 2: 1H-NMR-spectra comparison: liquor of ALS patient (upper curve) and liquor of healthy individual (lower curve), 2-hydroxybutyric acid (fourth line from below, triplett signal at about 0.90 and multiple signal at about 1.60-1.80), ethanol artefact from dip solution (third line from bottom, triplett signal at about 1.20).

FIG. 3: 1H-NMR-spectrum (urine) of patient 1 recorded in May 2008 (HMUR1605-d20 11/1 F:w HM d20 (1) 300/300), with control of a healthy donor.

FIG. 4: 1H-NMR-Spectrum (urine) of patient 2 recorded in August 2009 (SV-UR141009-d2o), first part.

FIG. 5: 1H-NMR-Spectrum (urine) of patient 2 recorded in August 2009 (SV-UR141009-d2o), second part.

### EXAMPLES

### 1. General

Samples from at least four patients, which have ALS according to the standard criteria ICD-10 (World Health Organisation), were examined for markers having significant variations from reference values, which were the respective markers (quantitatively and qualitatively) from healthy individuals of same age and sex). The samples were subjected to high frequency NMR (Bruker Avance 600 NMR spectrometer; Daltonics Esquire Mass spectrometer). In the following, the most significant variations are described.

### 2. Blood Count Analysis

A four week blood count was analysed as follows. Proteins were separated from 250 μl samples of blood or CSF with Sepack chromatography columns. 350 μl D₂O per sample were added. Samples were analysed by NMR combined with mass spectroscopy for low molecular metabolites. Other samples were prepared without protein separation and examined for protein and peptide markers. 250 μl urine samples were directly mixed with 350 μl D₂O and examined.

Glucose: the level was found to alternate between 48 and 120 mg/dl. The alternation is far too high compared to reference variations in healthy individuals. A glucose tolerance test (75 g glucose in 300 ml water in the morning sober at 10:26 am) however shows a relatively normal progress. For example, the following values were measured: 10:25 am (sober): 93 mg/dl; 11:25 am: 113 mg/dl; 12:25 am: 101 mg/dl.

Lactate: 5-10.8 mmol/l (reference 0.5-2.2 mmol/l). The value is far too high and indicates a significant lactate alkalosis with the respective complications (as outlined further below). The values for lactate and also citrate were also too high in the urine.

GSH/Glutathione: decreased from 3.0 to 1.8 μmol/l (reference 2.4-4.4 μmol/l)

Triglyceride: the values alternated between 208 and 347 mg/dl (reference 35-175 mg/dl.)

LDL/HDL-quotient: the value moved steadily downwards from 4.4 to 3.5 (reference <4).

IGF-1: the value alternated around a mean value of 130 ng/ml (reference 150-350 ng/ml).

ANA: the value decreased from 1:320 to 1:160 in the mean but was still too high (reference <1:80 titre).

CK-Nac decrease from 294 to 217 U/1 (reference <190 U/l).

Leucocytes: decrease from 11.7 to 9.7×10³/μl (reference 4-9.4×10³/μl).

Neutrophiles: decrease from 77 to 63% (reference 42-75%).

Monocytes: increase from 2.0 to 3.2% (reference 3.4-12%).

Erythrocytes: borderline fluctuation around 4.4×10⁶/μl (reference 4.6-6.2×10⁶/μl).

CD3/T-cells: increase from 340 to 394 (fluctuating), (reference 467-633 FI mean).

NK-cells: (reference 536-1056 FI mean) increasing from 340 to 394.

CD14/monocytes: increasing from 1090 to 1502 FI mean (reference 1372-1881 FI mean)

### 3. Liquor Analysis

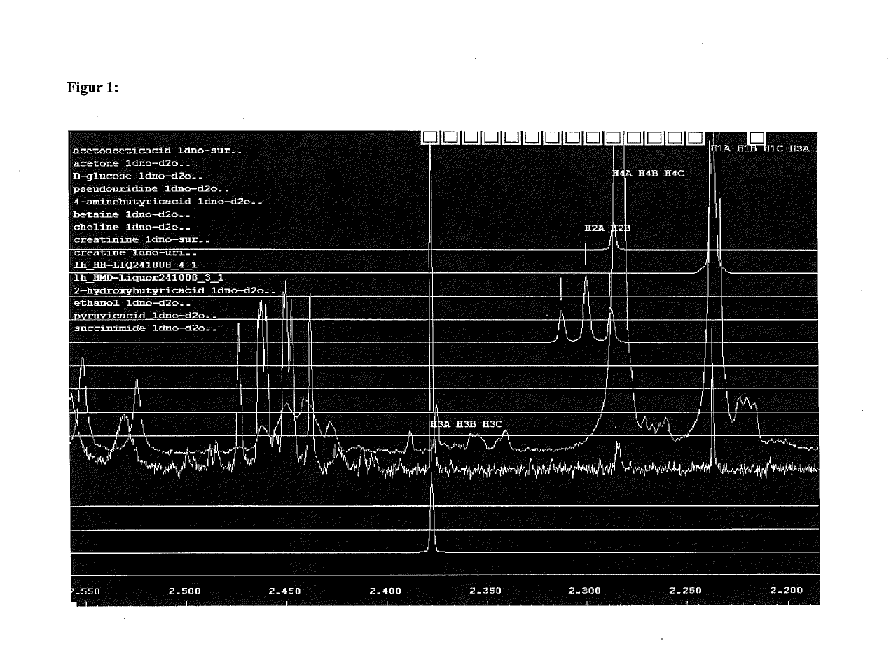

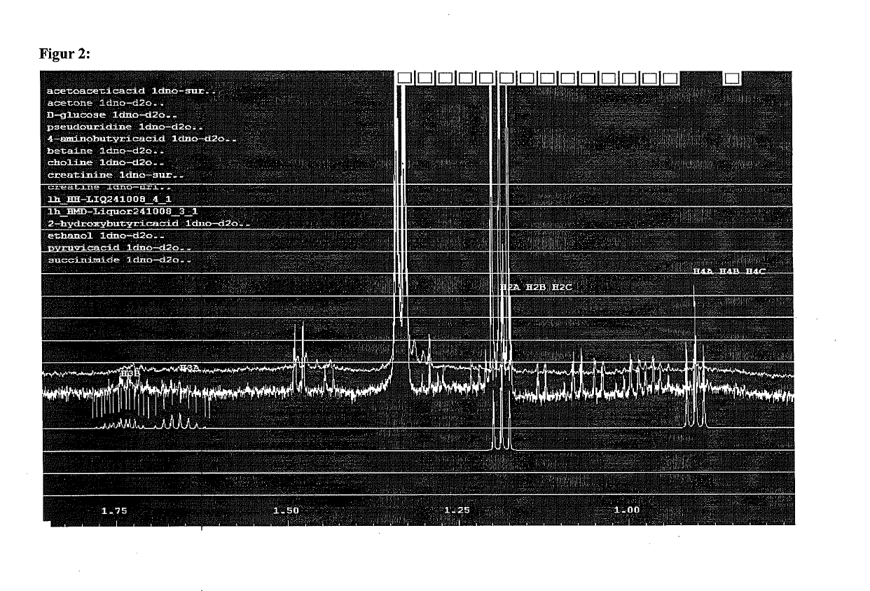

The liquor of an ALS patient and of healthy individuals (reference probe) were collected, pre-treated and examined as outlined above. Compared to normal values, a significant quantity of ketone bodies was found in the liquor of the patient. Furthermore, an aberrant ratio of glucose to lactate was observed. Comparable values can be found in blood and urine. Results are shown in FIGS. 1 and 2, in which the results of the two samples are overlaid and further combined with known markers. The results show significant differences in the ALS and reference probe. By means of the standards, significant peaks could be correlated to markers. The ALS patient liquor shows a strong increase in acetone and acetoacetate levels (FIG. 1) and a slight increase in hydroxybutyric acid, each compared to the reference.

### 4. Urine Analysis

The urine of two ALS patients was analysed and compared to a healthy individual. ALS has been diagnosed for patient 1 in December 2007. The ALSF-R Score was about 20 when samples were analyzed. ALS has been diagnosed for patient 2 around 1999. The ALSF-R score was about 2 when samples were analyzed. Samples of both patients were taken and studied approximately in parallel.

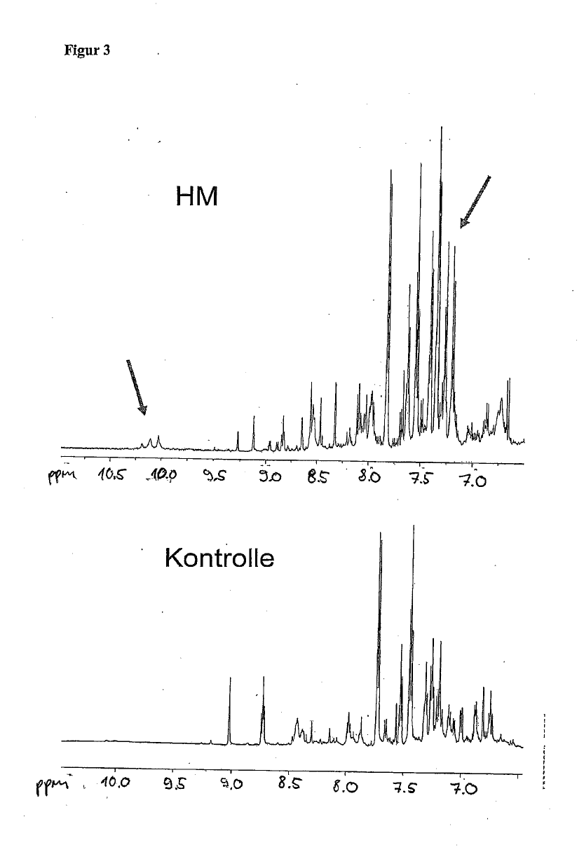

FIG. 3 shows a first 1H-NMR-spectrum (urine) recorded in May 2008 and a control of a healthy donor. Signals of at least 4 alkenales (nonenal etc.) were detected in the blood and urine of patient 1 on the one hand in the range of 10 ppm and aberrant identity patterns of the metabolites compared to the control show in the range of 7-9 ppm. Relevant signals for phenylalanine and metabolic substances associated therewith and histidine are also detected.

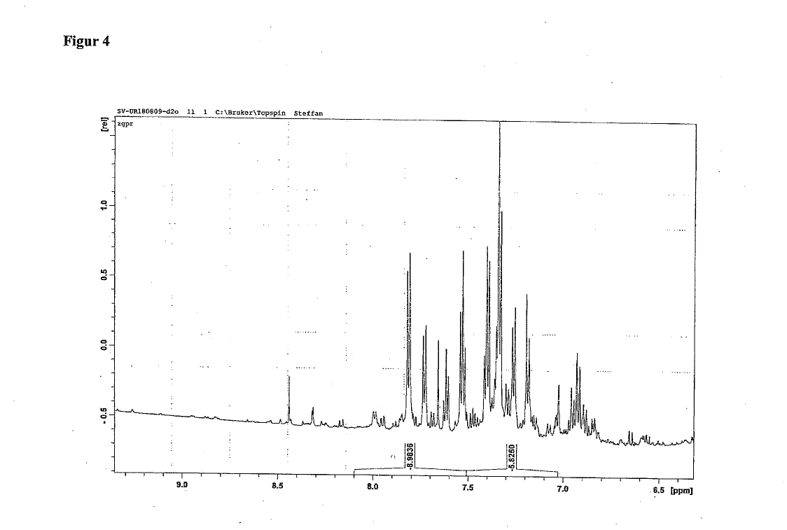

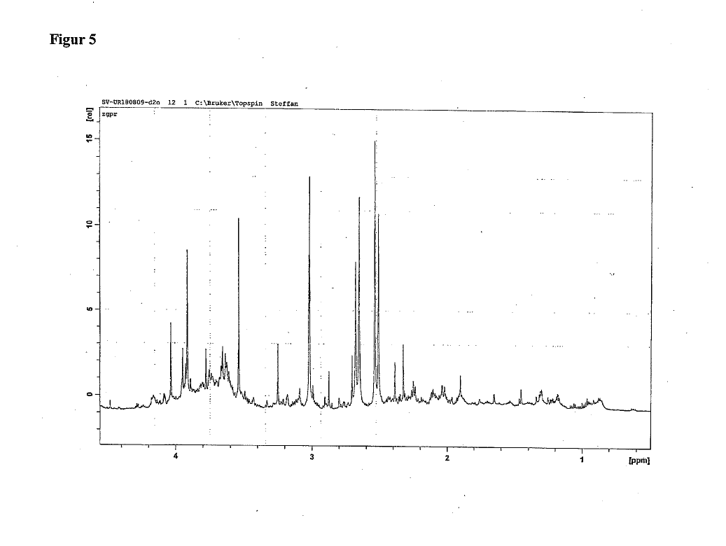

FIG. 4 shows the NMR-Spectrum (urine) of patient 2 recorded in August 2009 (SV-UR141009-d2o). A striking resemblance to the spectrum of the first patient of May 2008 (FIG. 3) was observed. The aromatic amino acids and heterocyclics are at the same level. In the aliphatic part, the ratio citrate to creatine is about 1 (FIG. 5).

### LITERATURE

Holmes et al., Metabolic Profiling of CSF: Evidence That Early Intervention May Impact on Disease Progression and Outcome in Schizophrenia. PLOS Medicine 2006, 3, 1420-1428.
Ilzecka J, Stelmasiak Z, Solski J, Wawrzycki S, Szpetnar M. Plasma amino acids percentages in amyotrophic lateral sclerosis patients, Neurol Sci 2003; 24: 293-95.
Mitchell, R. M., Freeman, W. M., Randazzo, W. T., Stephens, H. E., Beard, J. L., Simmons, Z., Connor, J. R. A CSF biomarker panel for identification of patients with amyotrophic lateral sclerosis, Neurology 2009; 72; 14-19.
Patten B M, Harati Y, Acosta L, Jung S S, Felmus M T. Free amino acid levels in amyotrophic lateral sclerosis. Ann Neurol 1978; 3: 305-09.
Petroff et al., High-Resolution Proton Magnetic Resonance Analysis of Human Cerebrospinal Fluid, J. of Neurochem. 1986, 47, 1270-1276.
Ranganathan S, Nicholl G C, Henry S, et al. Comparative proteomic profiling of cerebrospinal fluid between living and post mortem ALS and control subjects. Amyotroph Lateral Seler 2007; 8:373-79.
Turner, M. R., Kiernan, M. C., Leigh, P. N., Talbot, K., Biomarkers in amyotrophic lateral sclerosis, Lancet Neurol 2009; 8: 94-109.
Wevers et al., Standardized Method for High-Resolution ¹H-NMR of Cerebrospinal Fluid, Clin. Chem. 41, 744-751.

1. A method for the diagnosis of amyotrophic lateral sclerosis, comprising the steps of
providing a sample from a patient;
determining in the sample the level and/or activity of at least one marker, which is indicative of a glucose consumption disorder;
comparing the level and/or activity to a known standard; and
deducing whether the patient has, or is susceptible to, amyotrophic lateral sclerosis.

2. The method of claim 1, wherein the glucose consumption disorder is a ketonuria.

3. The method of claim 1, wherein the marker is a metabolic marker.

4. The method of claim 1, wherein the sample is cerebrospinal fluid (CSF), urine or blood, or a fraction of any of these samples.

5. The method of claim 1, wherein
the sample is cerebrospinal fluid (CSF) and the glucose consumption disorder is ketonuria, and/or
the sample is urine and the glucose consumption disorder is a phenylketonuria, and/or
the sample is blood and the glucose consumption disorder is a lactate acidose.

6. The method of claim 1, wherein the at least one markers is selected from the group consisting of ketone bodies, phenyl compounds which are not peptides, alkenales, catecholamines, adrenalin and noradrenalin, pyruvate, glucose, lactate, citrate, creatine, phosphocreatine, insulin, cortisol and glutathion.

7. The method of claim 1, wherein the sample is cerebrospinal fluid (CSF) and wherein the at least one marker is selected from the group consisting of ketone bodies, preferably acetone or acetylacetone, and cyclopropane.

8. The method of claim 1, wherein the sample is urine or blood and wherein the at least one marker is selected from the group consisting of phenyl compounds which are not peptides, phenylacetate, phenylpyruvate or phenyllactate, alkenales and pyruvate.

9. The method of claim 1, wherein the sample is blood or urine and wherein the marker is selected from the group consisting of catecholamines, adrenalin and noradrenalin, phenyl compounds which are not peptides, phenylalanine, glucose, lactate, citrate, creatine, phosphocreatine, insulin, cortisol and glutathion.

10. The method of claim 1, wherein in step (d) it is deduced that the patient has, or is susceptible to, amyotrophic lateral sclerosis, when the level and/or activity of the markers deviates at least 10% from the known standard.

11. The method of claim 1, further comprising
determining in the sample the level and/or activity of at least one protein and/or cell and/or the pH.

12. The method of claim 11, wherein the protein(s) or cell(s) are selected from the group consisting of ANA, CK-Nac, CD3/T-cells, LDL, HDL, leucocytes, neutrophiles, monocytes, erythrocytes and amyloid A.

13. The method of claim 1, wherein in step (b) the level of the metabolic marker is determined by 1H-NMR spectroscopy.

14. A method for the diagnosis of amyotrophic lateral sclerosis, comprising the steps of
providing a sample from a patient;
determining in the sample the level and/or activity of at least one metabolic marker;
comparing the level and/or activity to a known standard; and
deducing whether the patient has, or is susceptible to, amyotrophic lateral sclerosis, wherein the at least one marker is selected from the group consisting of acetone, acetoacetone, phenyl compounds which are not peptides, alkenales, pyruvate, catecholamines, adrenalin and noradrenalin, glucose, lactate, citrate, creatine, phosphocreatine, insulin, cortisol and glutathion.

15. The use of a method of of claim 1, further comprising monitoring the progression of ALS in a patient and/or monitoring an ALS therapy in a patient.

16. The method of claim 2, wherein the glucose consumption disorder is ketoacidosis.

17. The method of claim 5, wherein the sample is cerebrospinal fluid (CSF) and the glucose consumption disorder ketoacidosis, and/or
the sample is urine and the glucose consumption disorder is a phenylketonuria, and/or
the sample is blood and the glucose consumption disorder is a lactate acidose.

18. The method according to claim 6, wherein at least two markers are selected from the group consisting of ketone bodies, phenyl compounds which are not peptides, alkenales, catecholamines, adrenalin and noradrenalin, pyruvate, glucose, lactate, citrate, creatine, phosphocreatine, insulin, cortisol and glutathion.

19. The method of claim 6, wherein at least three markers are selected from the group consisting of ketone bodies, phenyl compounds which are not peptides, alkenales, catecholamines, adrenalin and noradrenalin, pyruvate, glucose, lactate, citrate, creatine, phosphocreatine, insulin, cortisol and glutathion.

20. The method of claim 1, wherein in step (d) it is deduced that the patient has, or is susceptible to, amyotrophic lateral sclerosis, when the level and/or activity of the markers deviates at least 20% from the known standard.
